SEQUENCE LISTING (18 sequences)

SEQ ID NO: 1 - DNA, 26 bp, Artificial Sequence
  single strand linear DNA 1
aggctgattcggttcatgcggatcca

SEQ ID NO: 2 - DNA, 26 bp, Artificial Sequence
  single strand linear DNA 2
tggatccgcatgacattcgccgtaag

SEQ ID NO: 3 - DNA, 26 bp, Artificial Sequence
  single strand linear DNA 3
cttacggcgaatgaccgaatcagcct

SEQ ID NO: 4 - DNA, 26 bp, Artificial Sequence
  single strand linear DNA 1 variant
aggctgattcggttcatgcggatcca

SEQ ID NO: 5 - DNA, 26 bp, Artificial Sequence
  single strand linear DNA 2 variant
tggatccgcatgacattcgccgtaag

SEQ ID NO: 6 - DNA, 26 bp, Artificial Sequence
  single strand linear DNA 3 variant
cttacggcgaatgaccgaatcagcct

SEQ ID NO: 7 - DNA, 26 bp, Artificial Sequence
  single strand linear DNA 2 variant
tggatccgcatgacattcgccgtaag

SEQ ID NO: 8 - DNA, 26 bp, Artificial Sequence
  single strand linear DNA 3 variant
cttacggcgaatgaccgaatcagcct

SEQ ID NO: 9 - DNA, 26 bp, Artificial Sequence
  single strand linear DNA 2 variant
tggatccgcatgacattcgccgtaag

SEQ ID NO: 10 - DNA, 26 bp, Artificial Sequence
  single strand linear DNA 3 variant
cttacggcgaatgaccgaatcagcct

SEQ ID NO: 11 - DNA, 30 bp, Artificial Sequence
  single strand linear DNA 3 variant
gactcttacggcgaatgaccgaatcagcct

SEQ ID NO: 12 - DNA, 30 bp, Artificial Sequence
  single strand linear DNA 1 variant
agtcaggctgattcggttcatgcggatcca

SEQ ID NO: 13 - DNA, 30 bp, Artificial Sequence
  single strand linear DNA 1 variant
gcataggctgattcggttcatgcggatcca

SEQ ID NO: 14 - DNA, 30 bp, Artificial Sequence
  single strand linear DNA 1 variant
atgcaggctgattcggttcatgcggatcca

SEQ ID NO: 15 - DNA, 26 bp, Artificial Sequence
  single strand linear DNA 2 variant
tggatccgcatgacattcgccgtaag

SEQ ID NO: 16 - DNA, 26 bp, Artificial Sequence
  single strand linear DNA 3 variant
cttacggcgaatgaccgaatcagcct

SEQ ID NO: 17 - DNA, 57 bp, Artificial Sequence
  single strand linear DNA 1 variant
gcgaggccagactgggaagaaatctgctcgcaggctgattcggttcatgcggatcca

SEQ ID NO: 18 - DNA, 58 bp, Artificial Sequence
  single strand linear DNA 1 variant
gcgagtcaacatcagtctgataagctactcgcaggctgattcggttcatgcggatcca

(19) United States
(12) Patent Application Publication
(10) Pub. No.: US 2015/0211056 A1
(43) Pub. Date: Jul. 30, 2015

(54) FUSION NANO LIPOSOME-FLUORESCENCE LABELED NUCLEIC ACID FOR IN VIVO APPLICATION, USES THEREOF AND PREPARATION METHOD THEREOF

(71) Applicant: RESEARCH & BUSINESS FOUNDATION SUNGKYUNKWAN UNIVERSITY, Suwon-si (KR)

(72) Inventors: Soong Ho UM, Suwon-si (KR); Kyung Soo PARK, Seoul (KR)

(73) Assignee: RESEARCH & BUSINESS FOUNDATION SUNGKYUNKWAN UNIVERSITY, Suwon-si (KR)

(21) Appl. No.: 14/607,902

(22) Filed: Jan. 28, 2015

(30) Foreign Application Priority Data
Jan. 29, 2014 (KR) 10-2014-0011290

(51) Int. Cl. C12Q 1/68

(52) U.S. Cl. CPC C12Q 1/6818; C12Q 1/6834

(57) ABSTRACT

The present disclosure relates to a fusion nano liposome-fluorescence labeled nucleic acid in which a bead having a surface binding with a branch-shaped nucleic acid structure labeled with a fluorophore or a branch-shaped nucleic acid structure having a hairpin loop end is included in an inside of a liposome, and a diagnosis application thereof. The fusion nano liposome-fluorescence labeled nucleic acid, or fusion nano liposome-fluorescence labeled hairpin loop structured nucleic acid may sense an external or internal signal, and high-sensitive diagnosis is possible even when mRNA and miRNA which is present at a low concentration in cells being targeted. Further, various target materials expressed inside and outside of a cell membrane may be targeted, and thus even a type of cancer which is hard to diagnose such as triple negative breast cancer also be flexibly diagnosed. Further, using various fluorophores, multiple cancer may be diagnosed at the same time.

### CROSS-REFERENCE TO RELATED APPLICATION

This application claims priority to and the benefit under 35 U.S.C. §119(a) of Korean Patent Application No. 10-2014-0011290, filed on Jan. 29, 2014 in the Korean Intellectual Property Office, the disclosure of which is incorporated herein by reference in its entirety for all purposes.

### STATEMENT REGARDING GOVERNMENT RIGHTS

This invention was made with government support of the Republic of Korea under Contract Nos. 2013R1A1A1058670, HI14C3301, and 2013R1A1A2016781 awarded by Korean Ministry of Science, ICT and Future Planning, Ministry of Health and Welfare, and Korean Ministry of Science, ICT and Future Planning. The government has certain rights in the invention.

### BACKGROUND

1. Field

The present disclosure relates to a fusion nano liposome-fluorescence labeled nucleic acid in which a polystyrene or silica bead, having a surface binding with a branch-shaped nucleic acid structure labeled with a fluorophore or a branch-shaped nucleic acid structure having a hairpin loop end, is included in an inside of a liposome; an imaging system using the fusion nano liposome-fluorescence labeled nucleic acid to diagnose disease; a disease-diagnosing method; and a method of producing the fusion nano liposome-fluorescence labeled nucleic acid.

2. Description of Related Art

Currently, a metal, a polymer, or the like which are harmful to human bodies are used for most diagnoses of cancer, and a definite diagnosis of cancer needs at least three days and up to one month or more. Further, since most diagnostic substances do not generate light in a wavelength range of visible light, there is a limitation to diagnose cancer visually until the cancer considerably progresses and a tumor is formed. Further, performing diagnosis in a living body in real time is also limited due to the same reason.

Accordingly, recent studies have been made with respect to a diagnostic substance which is made of bio-friendly materials to minimize harmful effects to a human body. Nucleic acids are materials which mostly receive attention, and being produced in various shapes through nano biotechnology. The technique in which fluorophores are bound to the nucleic acid nanostructures produced as described above is also drawing attention.

However, since nucleic acids are materials native to the living body, when inserted into the living body, such nucleic acids are likely to be broken by enzymes, and lose their original function. Further, a process of binding the diagnostic substance to a target material is additionally required to minimize non-specific binding of the diagnostic substance. However, a study through which all the above-described requirements are satisfied has not yet been performed up to now. Further, there are difficulties in diagnosing cancer cells such as triple negative breast cancer or the like in which a specific target is not expressed.

Accordingly, a high performance diagnostic substance for cancer which may effectively, harmlessly, and rapidly diagnose cancer is needed.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, a fusion nano liposome-fluorescence labeled nucleic acid for diagnosis is provided, in which a polystyrene or silica bead having a surface binding with a branch-shaped nucleic acid structure labeled with a fluorophore is included in an inside of a liposome.

The nucleic acid structure may have a shape in which linear nucleic acids selected from the group consisting of a base sequence of SEQ ID NO: 1 to 3 are bound to be in a Y-branched shape.

The linear nucleic acid may further include a fluorophore at a 5′ end.

The fluorophore may be selected from the group consisting of fluorescein, Texas Red, rhodamine, alexa, cyanine, BODIPY, and coumarin.

In another general aspect, a fusion nano liposome-fluorescence labeled hairpin loop structured nucleic acid for diagnosis is provided, in which a polystyrene or silica bead having a surface binding with a branch-shaped nucleic acid structure having a hairpin loop end is included in an inside of a liposome.

The hairpin loop end may include a base sequence having a complementary sequence with a target ribonucleic acid (RNA).

The nucleic acid structure may be further labeled with a fluorophore and a quencher.

The fluorophore may be selected from the group consisting of fluorescein, Texas Red, rhodamine, alexa, cyanine, BODIPY, and coumarin.

The quencher may be selected from the group consisting of TAMRA, BHQ, Iowa Black RQ, and a molecular grove binding non-fluorescence quencher (MGBNFQ).

The branch-shaped nucleic acid structure having a hairpin loop end may have a shape in which linear nucleic acids are bound to be in a Y-branched shape, and one or more of the linear nucleic acids form a hairpin loop end.

The liposome may be formed of a cationic lipid including DOTAP (1,2-dioleoyl-3-trimethylammonium-propane) and cholesterol as constituents.

The liposome may be formed of a neutral lipid including DOPC (1,2-dioleoyl-sn-glycero-3-phosphocholine), 18:1 PEG2000 PE (1,2-dioleoyl-sn-glycero-3-phosphoethanolamine-N-[methoxy (polyethylene glycol)-2000]), DOPE (1,2-dioleoyl-sn-glycero-3-phosphoethanolamine), and cholesterol as constituents.

A bio-diagnostic imaging system may include the fusion nano liposome-fluorescence labeled nucleic acid.

A bio-diagnostic imaging system may include the fusion nano liposome-fluorescence labeled (hairpin loop structured) nucleic acid.

The system may measure fluorescence in cells.

In another general aspect, a method of producing a fusion nano liposome-fluorescence labeled nucleic acid for diagnosis, may include the following steps: a) preparing a Y-branch-shaped nucleic acid structure with linear nucleic acids which respectively have a fluorophore, biotin, or cohesive end at a 5′ end using an annealing method; b) binding the nucleic acid structure to a streptavidin-coated surface of a polystyrene or silica bead to prepare a fluorescence-labeled nucleic acid nanosphere; and c) mixing a solution containing the fluorescence-labeled nucleic acid nanosphere, and a solution containing a liposome formed of a cationic lipid or neutral lipid.

In another general aspect, a method of producing a fusion nano liposome-fluorescence labeled hairpin loop structured nucleic acid for diagnosis, may include the following steps: a) preparing a Y-branch-shaped nucleic acid structure with linear nucleic acids which respectively have a fluorophore and quencher, biotin, or cohesive end at a 5′ end using an annealing method, wherein one or more of the linear nucleic acids include a base sequence having a complementary sequence with a target RNA, and a base sequence forming a hairpin loop end at the 5′ end; b) binding the hairpin loop structured nucleic acid structure to a streptavidin-coated surface of a polystyrene or silica bead to prepare a fluorescence labeled hairpin loop structured nucleic acid nanosphere; and c) mixing a solution containing the fluorescence labeled hairpin loop structured nucleic acid nanosphere, and a solution containing a liposome formed of a cationic lipid or neutral lipid.

The linear nucleic acid may be selected from the group consisting of a base sequence of SEQ ID NO: 1 to 16.

The linear nucleic acid may be selected from the group consisting of a base sequence of SEQ ID NO: 17 or 18.

The fluorophore may be selected from the group consisting of fluorescein, Texas Red, rhodamine, alexa, cyanine, BODIPY, and coumarin.

The quencher may be selected from the group consisting of TAMRA, BHQ, Iowa Black RQ, and a molecular grove binding non-fluorescence quencher (MGBNFQ).

The liposome formed of a cationic lipid may be prepared by mixing DOTAP (1,2-dioleoyl-3-trimethylammonium-propane) and cholesterol in the mass ratio of 6:4 to 9:1.

The liposome formed of a neutral lipid may be prepared by mixing DOPC (1,2-dioleoyl-sn-glycero-3-phosphocholine), 18:1 PEG2000 PE (1,2-dioleoyl-sn-glycero-3-phosphoethanolamine-N-[methoxy(polyethylene glycol)-2000]), DOPE (1,2-dioleoyl-sn-glycero-3-phosphoethanolamine), and cholesterol in the mass ratio of 12:1:1:6 to 14:1:1:1.

The method further may include preparing a branch-shaped nucleic acid structure by connecting a cohesive end of a Y-branch-shaped nucleic acid structure to another Y-branch-shaped nucleic acid structure using a T4 ligase after step a).

A solution containing the sphere and a solution containing the liposome may be mixed in the volume ratio of 1:1 to 1:4 in step c).

In another general aspect, a method of diagnosing disease may include a step of injecting the fusion nano liposome-fluorescence labeled (hairpin loop structured) nucleic acid to a subject requiring diagnosis of disease, and measuring fluorescence.

The disease may be cancer.

The injecting may be performed through an oral administration, an intravenous injection, an intraperitoneal injection, an intramuscular injection, an intra-arterial injection, or a hypodermic injection.

Other features and aspects will be apparent from the following detailed description, the drawings, and the claims.

### BRIEF DESCRIPTION OF THE DRAWINGS

FIG. 1 is a diagram showing a model of a fusion nano liposome-fluorescence labeled nucleic acid and a formed lipid layer, and a structural formula of lipids, cholesterols, nucleic acid structures, and ligands;

FIGS. 2A to 2D show (a) a Y-shaped nucleic acid nanostructure, (b) a Y-shaped nucleic acid structure having a hairpin loop end, (c) a branch-shaped nucleic acid nanostructure, and (d) a result of determination through electrophoresis after synthesizing the structures of the (a) to (c);

FIG. 3 shows photographs using a confocal microscope after a nucleic acid nanostructure is bound to a silica bead to concentrate a signal of a fusion nano fluorescence labeled nucleic acid of an encoded nucleic acid nanostructure;

FIG. 4 is a graph showing a result of analysis using a flow cytometer that a signal intensity is adjusted according to control of an amount of the used fusion nano fluorescence labeled nucleic acid after a nucleic acid nanostructure is bound to a silica bead to concentrate a signal of a fusion nano fluorescence labeled nucleic acid of an encoded nucleic acid nanostructure;

FIG. 5 is a graph showing a result of analysis through dynamic light scattering (DLS) of a size and controllability of a surface charge according to a liposome composition of the fusion nano liposome-fluorescence labeled nucleic acid.

FIG. 6 is a graph showing a result of comparison using a flow cytometer after breast cancer cells (MCF-7) and ovarian carcinoma cells (SK-OV-3) are treated with a fusion nano liposome-fluorescence labeled nucleic acid to which a luteal hormone releasing hormone targeted protein is bound;

FIGS. 7A and 7B shows (a) a graph of a result of analysis of breast cancer cells (MCF-7) and normal mammary cells (MCF-10A) which are treated with a fusion nano liposome-fluorescence labeled nucleic acid including a hairpin loop structured nucleic acid nanostructure for diagnosing a messenger ribonucleic acid (EZH2) (left side) or including a normal Y-shaped nucleic acid nanostructure (right side), and (b) a result graph showing a signal intensity due to a Forster resonance energy transfer effect with respect to an absolute quantity of a signal of the fusion nano liposome-fluorescence labeled nucleic acid introduced into cells through two signals, in which overexpression levels of messenger ribonucleic acids in cancer cells and normal cells are compared; and

FIGS. 8A and 8B show graphs of analysis using a flow cytometer after breast cancer cells (MCF-7) and other breast cancer cells (SK-BR-3) are treated with a fusion nano liposome-fluorescence labeled nucleic acid including both of a hairpin loop structured nucleic acid nanostructure for diagnosing a micro ribonucleic acid (miR21) and a normal Y-shaped nucleic acid nanostructure having green fluorophores at two ends, where (a) is a graph of analyzing a signal of a hairpin loop structured nucleic acid nanostructure (left side) and a signal of a green fluorophore (right side), and (b) is a graph of comparing an overexpression level of the micro ribonucleic acid between cancer cells through a relative ratio of the two signals.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

Exemplary embodiments of the present disclosure will be described in detail below with reference to the accompanying drawings. While the present disclosure is shown and described in connection with exemplary embodiments thereof, it will be apparent to those skilled in the art that various modifications can be made without departing from the spirit and scope of the present disclosure.

The following detailed description is provided to assist the reader in gaining a comprehensive understanding of the methods, apparatuses, and/or systems described herein. However, various changes, modifications, and equivalents of the systems, apparatuses and/or methods described herein will be apparent to one of ordinary skill in the art. The progression of processing steps and/or operations described is an example; however, the sequence of and/or operations is not limited to that set forth herein and may be changed as is known in the art, with the exception of steps and/or operations necessarily occurring in a certain order. Also, descriptions of functions and constructions that are well known to one of ordinary skill in the art may be omitted for increased clarity and conciseness.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

The present inventors have studied the development of a cancer cell-diagnostic substance allowing for high-sensitive multiple detection of cancer cells and in vivo application thereof, and as a result, the present disclosure was completed.

Accordingly, the present disclosure is directed to providing a fusion nano liposome-fluorescence labeled nucleic acid for diagnosis, in which a polystyrene or silica bead having a surface that binds with a branch-shaped nucleic acid structure labeled with a fluorophore is included in an inside of a liposome. Also, the present disclosure is directed to providing a fusion nano liposome-fluorescence labeled hairpin loop structured nucleic acid for diagnosis, in which a polystyrene or silica bead having a surface that binds with a branch-shaped nucleic acid structure having a hairpin loop end is included in an inside of a liposome.

Further, the present disclosure may provide a method of producing a fusion nano liposome-fluorescence labeled nucleic acid for diagnosis, including the following steps:

a) preparing a Y-branch-shaped nucleic acid structure with linear nucleic acids which respectively have a fluorophore, biotin, or cohesive end at a 5′ end using an annealing method;

b) binding the nucleic acid structure to a streptavidin-coated surface of a polystyrene or silica bead to prepare a fluorescence-labeled nucleic acid nanosphere; and

c) mixing a solution containing the fluorescence-labeled nucleic acid nanosphere, and a solution containing a liposome formed of a cationic lipid or neutral lipid in the volume ratio of 1:1 to 1:4.

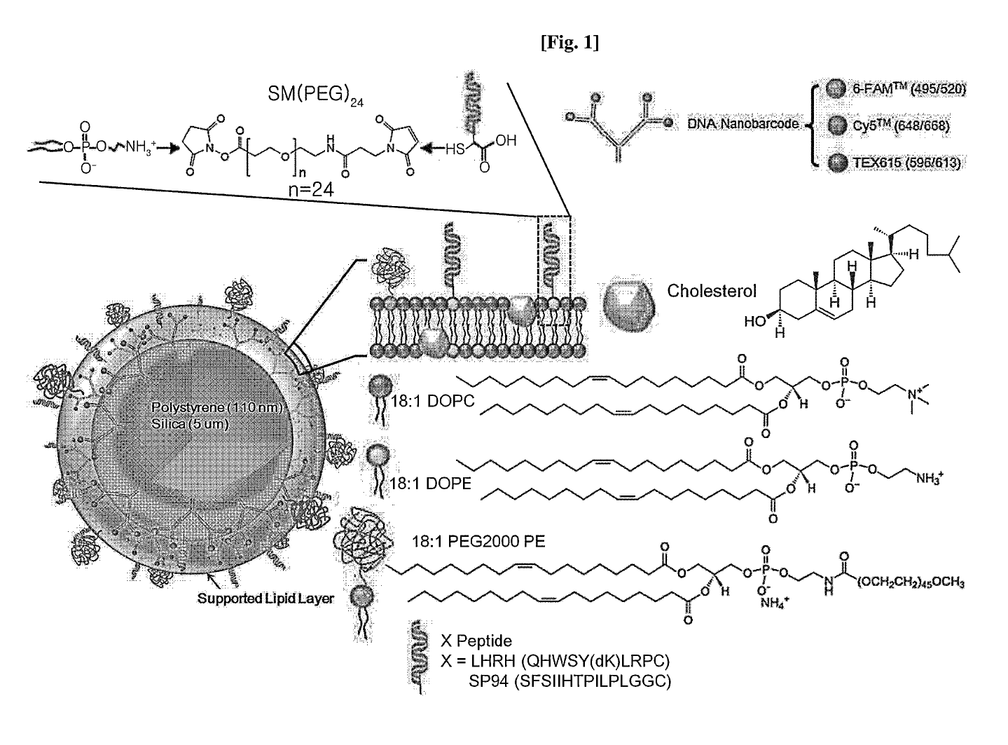

With regard to this, the fusion nano liposome-fluorescence labeled nucleic acid according to an embodiment of the present disclosure may be used to diagnose disease, and in the embodiment of the present disclosure, a nucleic acid structure labeled with a fluorophore is bound to a silica or polystyrene bead, and then a surface of the bead is coated with a supported lipid bilayer to be implemented in cells (refer to FIG. 1). Here, although the sphere is prepared using silica having a micro-scale size to easily determine whether a system works or not through a microscope, in an embodiment of the present disclosure, the fusion nano liposome-fluorescence labeled nucleic acid may be prepared using a polystyrene bead having a nano-scale size. However, any bead having a nano size capable of being used in a living body may be used without limitation. A size of the prepared fusion nano liposome-fluorescence labeled nucleic acid may be properly adjusted when the size and shape of the fusion nano liposome-fluorescence labeled nucleic acid may allow the fusion nano liposome-fluorescence labeled nucleic acid to be injected to the body. Further, the fusion nano liposome-fluorescence labeled nucleic acid may be prepared to be a sphere having a diameter in a range of 100 to 300 nm

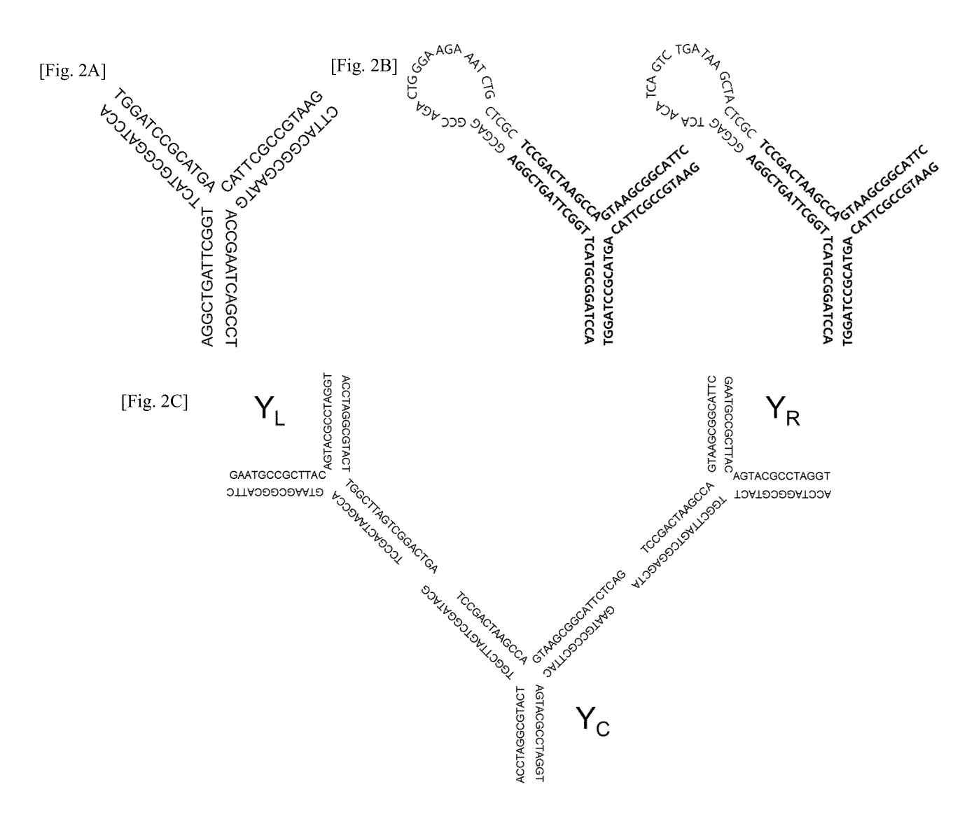

In an embodiment of the present disclosure, the nucleic acid structure may be prepared to have a branched shape by connecting the nucleic acid structures in which linear nucleic acids are bound to be in a Y-branched shape or linear nucleic acids having cohesive ends are bound to be in a Y-branched shape using a T4 ligase such that the cohesive ends are bound to each other. Since the branch shaped nucleic acid structure has at least three of 5′ ends, the 5′ ends may be labeled with fluorophores, and thus the nucleic acid structure may be used as a fluorescence barcode (refer to FIG. 2).

Further, in order to detect an internal signal of cells, the nucleic acid structure may have a Y-branch shape prepared using linear nucleic acids including a complementary sequence with a target ribonucleic acid and a short sequence which allows binding with a hairpin loop structured nucleic acid structure, such that the nucleic acid structure may bind with a target messenger ribonucleic acid (mRNA) or a target micro ribonucleic acid (miRNA). Here, the linear nucleic acids including a complementary sequence with the target ribonucleic acid are further labeled with fluorophores and quenchers. Accordingly, the hairpin loop structured nucleic acid structure which binds to the target ribonucleic acid works based on a Forster resonance energy transfer effect (FRET) between the fluorophores and quenchers. That is, when the hairpin loop structured nucleic acid structure does not bind to the target ribonucleic acid, light is not generated due to the FRET, and when the hairpin loop structured nucleic acid structure binds to the target ribonucleic acid, light is generated from fluorophores.

Accordingly, the present disclosure may provide a method of producing a fusion nano liposome-fluorescence labeled hairpin loop structured nucleic acid for diagnosis, including the following steps:

a) preparing a Y-branch-shaped nucleic acid structure with linear nucleic acids which respectively have a fluorophore and quencher, biotin, or cohesive end at a 5′ end using an annealing method, wherein one or more of the linear nucleic acids include a base sequence having a complementary sequence with a target RNA, and a base sequence forming a hairpin loop end at the 5′ end;

b) binding the hairpin loop structured nucleic acid structure to a streptavidin-coated surface of a polystyrene or silica bead to prepare a fluorescence labeled hairpin loop structured nucleic acid nanosphere; and

c) mixing a solution containing the fluorescence labeled hairpin loop structured nucleic acid nanosphere, and a solution containing a liposome formed of a cationic lipid or a neutral lipid in the volume ratio of 1:1 to 1:4.

Here, the production method is not limited to the above-described steps, and an order and/or composition of the steps may be properly modified as long as the fusion nano liposome-fluorescence labeled nucleic acid according to an embodiment of the present disclosure may be produced.

In the 5′ end including a base sequence having a complementary sequence with the target RNA and a base sequence forming a hairpin loop end, the base sequence forming a hairpin loop end may be referred to as a “stem”, and the 5′ end may be designed by binding the stems having complementary sequences with each other to both ends of the base sequence having a complementary sequence with the target RNA, respectively. For example, complementary base sequences such as GCGAG and CTCGC may be bound to both ends of the base sequence having a complementary sequence with the target RNA to form a hairpin loop structure. The stem part may be maintained even when the target RNA is modified, but is not necessarily required to have a specific sequence, and a length of the stem part may also be arbitrarily designed. However, it is preferable to design the stem sequence such that GC contents are maintained to be 70 to 80% with respect to AT contents, and to design such that thermodynamic energy to maintain a loop is lower than thermodynamic energy to break a loop by binding a target nucleic acid to a hairpin loop structured nucleic acid.

In an embodiment of the present disclosure, the fluorophore may be preferably fluorescein, Texas Red, rhodamine, sulfonated fluorescent dyes (such as Alexa Fluor), cyanine, boron-dipyrromethene (BODIPY), or coumarin, and more preferably, may be 6-FAM, Texas 615, Alexa Fluor 488, Cy5, or Cy3. The quencher may be preferably TAMRA, BHQ, Iowa Black RQ, or a molecular grove binding non-fluorescence quencher (MGBNFQ), and more preferably, may be Iowa Black RQ. However, the fluorophore and quencher are not limited thereto, and those skilled in the art may properly modify and use any fluorophore or quencher capable of being used in the living body.

The lipid composition of the liposome according to an embodiment of the present disclosure may be changed to adjust interaction with cells according to a desired use. In an embodiment of the present disclosure, the liposome was prepared with a lipid having a positive charge such that the fusion nano liposome-fluorescence labeled nucleic acid according to the embodiment of the present disclosure may flow into a cytoplasm by fusing with the cell membrane non-specifically to the target cell. The lipid having a positive charge may be preferably prepared by mixing DOTAP (1,2-dioleoyl-3-trimethylammonium-propane) and cholesterol in the mass ratio of 6:4 to 9:1, and more preferably, the lipid having a positive charge may be prepared by mixing DOTAP and cholesterol in the mass ratio of 7:3 as in an embodiment of the present disclosure, but is not limited thereto. Further, in another embodiment of the present disclosure, the liposome is prepared with a lipid having a neutral charge to prevent the fusion nano liposome-fluorescence labeled nucleic acid according to the embodiment of the present disclosure from non-specifically binding to types of cells. The lipid having a neutral charge may be preferably prepared by mixing DOPC (1,2-dioleoyl-sn-glycero-3-phosphocholine), 18:1 PEG2000 PE (1,2-dioleoyl-sn-glycero-3-phosphoethanolamine-N-[methoxy(polyethylene glycol)-2000]), DOPE (1,2-dioleoyl-sn-glycero-3-phosphoethanolamine), and cholesterol in the mass ratio of 12:1:1:6 to 14:1:1:1, and more preferably, the lipid having a neutral charge may be preferably prepared by mixing DOPC, 18:1 PEG2000 PE, DOPE, and cholesterol in the mass ratio of 12:1:1:6 as in the embodiment of the present disclosure, but is not limited thereto. Further, the liposome may be prepared without cholesterol.

Further, the fusion nano liposome-fluorescence labeled nucleic acid according to an embodiment of the present disclosure may be prepared by mixing the same solutions (solvents) to which a fluorescence labeled (hairpin loop structured) nucleic acid sphere and a liposome are added respectively. The solution containing the fluorescence labeled (hairpin loop structured) nucleic acid sphere and the solution containing the liposome may be preferably mixed in the volume ratio of 1:1 to 1:4. Most preferably, the solution containing the fluorescence labeled (hairpin loop structured) nucleic acid sphere and the solution containing the liposome may be mixed in the volume ratio of 1:3 as in the embodiment of the present disclosure, but is not limited thereto. Here, the same solutions (solvents) should be used to prevent a destruction of the liposome by an osmotic pressure, and the solution may include distilled water or a phosphate solution such as phosphate buffer saline (PBS), but is not limited thereto.

The fusion nano liposome-fluorescence labeled nucleic acid according to an embodiment of the present disclosure is configured such that nucleic acids are added to ends of the Y-shaped nucleic acid structure, labeled with fluorophores having various tones, and thus nucleic acid structures having various shapes may be produced and various codes may be embodied.

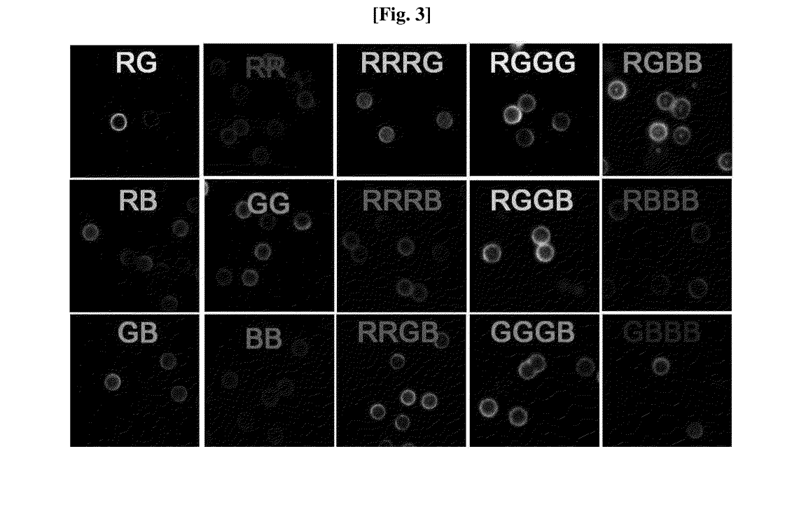

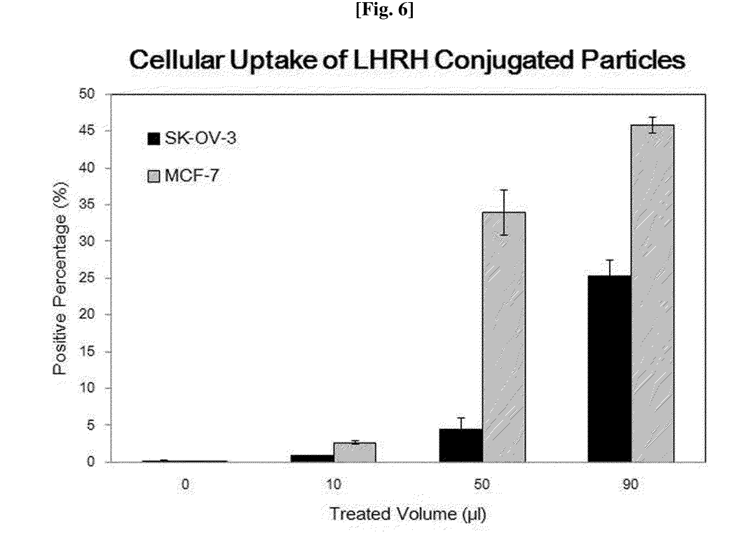

An embodiment of the present disclosure may detect an internal signal of cancer cells. A base sequence and a fluorophore are properly modified to design a linear nucleic acid, fluorescence labeled nucleic acid spheres having various shapes are produced using the linear nucleic acids (refer to FIG. 3), and the fluorescence labeled nucleic acid sphere was proven to be capable of embodying various codes (refer to FIG. 3). Further, cancer cells were treated with the prepared fusion nano liposome-fluorescence labeled (hairpin loop structured) nucleic acid, a fluorescence value was measured, and consequently, the fusion nano liposome-fluorescence labeled nucleic acid according to the embodiment of the present disclosure was determined to be successfully introduced into the cancer cells and emit fluorescence with an excellent signal intensity (refer to FIGS. 6 to 8). Here, in the prepared fusion nano liposome-fluorescence labeled (hairpin loop structured) nucleic acid, a targeted protein which targets the cancer cells may be connected to a surface of the liposome formed of a neutral lipid by a linker, and more fluorescence labeled hairpin loop structured nucleic acid structures capable of binding with the target RNA may be included in the fusion nano liposome-fluorescence labeled (hairpin loop structured) nucleic acid. The linker may be preferably (SM(PEG)₂₄), but is not limited thereto.

Accordingly, the fusion nano liposome-fluorescence labeled (hairpin loop structured) nucleic acid according to an embodiment of the present disclosure may be used as a bio-diagnostic imaging system, and in vivo images may be transferred by measuring fluorescence in cells using a flow cytometer or confocal microscope in the system.

In addition, the present disclosure may provide a disease-diagnosing method including a step of injecting the fusion nano liposome-fluorescence labeled (hairpin loop structured) nucleic acid to the individual requiring for diagnosis of disease and measuring fluorescence in cells. The injection may be performed through an oral administration, an intravenous injection, an intraperitoneal injection, an intramuscular injection, an intra-arterial injection, or a hypodermic injection method. The individual means a subject requiring diagnosis of disease, and more specifically, includes humans, or mammals such as primates which are non-human, mice, rats, dogs, cats, horses, cows or the like. Further, in the diagnosing method, since nucleic acid sequences may be modified to prepare the fusion nano liposome-fluorescence labeled (hairpin loop structured) nucleic acid according to diseases in need of diagnosis, the disease is not limited, the diagnosing method may be preferably intended to diagnose cancer, and most preferably, may be intended to diagnose breast cancer as in an embodiment of the present disclosure.

Further, an inside of the fusion nano liposome-fluorescence labeled (hairpin loop structured) nucleic acid according to an embodiment of the present disclosure may be empty. Thus the fusion nano liposome-fluorescence labeled (hairpin loop structured) nucleic acid may be prepared to include various materials. The material which may be included in the fusion nano liposome-fluorescence labeled (hairpin loop structured) nucleic acid is not limited, but a medicine may be preferably included in the fusion nano liposome-fluorescence labeled (hairpin loop structured) nucleic acid, and thereby the fusion nano liposome-fluorescence labeled (hairpin loop structured) nucleic acid may be used in a system for both bio-diagnostic imaging and treating.

Hereinafter, exemplary examples will be described to help understanding of the present disclosure. However, the following examples are merely provided for easy understanding of the present disclosure, and contents of the present disclosure are not limited to the following examples.

### Example 1

### Preparation of Fluorescence Labeled Nucleic Acid Sphere

To prepare a fluorescence labeled nucleic acid nanosphere as schematically shown in FIG. 1, a Y-shaped fluorescence labeled nucleic acid structure was prepared by annealing linear nucleic acids. For this, three types of single strand linear nucleic acids having complementary sequences with each other were designed and prepared (synthesis was consigned to Integrated DNA Technologies, Inc.) (refer to Table 1). The prepared linear nucleic acids were dissolved to Tris/EDTA buffers (TE buffers), each concentration of the TE buffers was calculated by measuring absorbance, and then the three types of the linear nucleic acids with the same number of moles were mixed. Then, double-screwed nucleic acid structures having various shapes were prepared through annealing, and the results are shown in FIGS. 2A to 2D. As shown in FIG. 2A, showing SEQ ID NO: 1 to 3, a fluorophore (dye) or biotin was bound to each 5′ end of the nucleic acid structures to prepare Y-shaped fluorescence labeled nucleic acid structures (see also SEQ ID NO: 4-18). Cohesive end sequences were added to linear nucleic acids to prepare Y-shaped nucleic acid structures, and the prepared Y-shaped nucleic acid structures were connected to prepare branch-shaped nucleic acid structures as shown in FIG. 2C, showing SEQ ID NO: 2, 3, and 20-23. Further, Y-shaped nucleic acid structures having a hairpin loop end were prepared using linear nucleic acids in which a DNA sequence including a sequence forming a loop (SEQ ID NO: 19 in conjunction with SEQ ID NO: 1 to 3) on both sides of the complementary sequence with the target RNA in cells is added to an end as shown in FIG. 2B, and the nucleic acid structures were determined to be properly prepared through electrophoresis as shown in FIG. 2D.

More specifically, in the method of producing the branch-shaped (fluorescence labeled) nucleic acid structure, three types of Y-shaped fluorescence labeled nucleic acid structures were prepared (Y_{L}, Y_{C}, and Y_{R}). The Y-shaped structures include cohesive ends having complementary sequences with each other at ends were prepared respectively first for a ligation between the Y-shaped nucleic acid structures, the three types of the structures with the same number of moles were mixed in one batch, and then were bound to each other using a T4 ligase at room temperature for 3 hours. FIG. 2C shows Y_{L} (SEQ ID NO: 2, 3, and 20), Y_{C} (SEQ ID NO: 2, 21, and 22), and Y_{R} (SEQ ID NO: 2, 3, and 23),

Further, to detect an internal signal of cancer cells, (fluorescence labeled) nucleic acid structures were prepared having a hairpin loop end which may complementarily bind to EZH2 messenger ribonucleic acid (mRNA) or miR21 micro ribonucleic acid (miRNA) in cells, which is a target RNA and may be detected. For this, Y-shaped nucleic acid structures were prepared using linear nucleic acids (refer to SEQ ID NO: 17 and 18 in Table 1) including a complementary sequence with the ribonucleic acid and a short sequence which binds the ribonucleic acid in a hairpin loop structure. Here, to determine the binding of the target ribonucleic acid hairpin loop structured nucleic acid structure, a fluorophore and a quencher were bound to both ends of the hairpin loop structured nucleic acid structure respectively to prepare a Y-shaped fluorescence labeled nucleic acid structure, such that light is not generated due to Forster resonance energy transfer effect between the fluorophore and quencher when the structure does not bind to the target ribonucleic acid, and when the structure binds to the target ribonucleic acid, the effect is removed and light is generated from the fluorophore. Further, the fluorescence labeled hairpin loop structured nucleic acid structure which may detect various targets may be prepared by designing an end DNA sequence of the Y-shaped nucleic acid structure according to the target, and arbitrarily modifying/adding fluorophores.

FIG. 2A is constituted of SEQ ID NO: 1 to 3. SEQ ID NO: 1 can be substituted with SEQ ID NO: 4, while SEQ ID NO: 2 and SEQ ID NO: 3 can be substituted with their respective modified versions; eg. 6-FAM, Cy5, or TEX615 conjugated Oligos (SEQ ID NO: 5-8, 15 and 16).

FIG. 2B is constituted of SEQ ID NO: 2, 3 and 17 (or 18). SEQ ID NO: 2 can be substituted with SEQ ID NO: 9, while SEQ ID NO: 3 can be substituted with any one of SEQ ID NO: 6, 8 and 16.

SEQ ID NO: 11 to 14 are included for creating tree-like DNA nanostructure of FIG. 2C. For the preparation of Y_{L}, SEQ ID NO: 2 and 3 (and their respective modified versions; eg. 6-FAM, Cy5, or TEX615 conjugated Oligos (SEQ ID NO: 5-8, 15 and 16)) and 12 were used. For the preparation of Yc, SEQ ID NO: 2 (and its respective modified version; eg. Biotinylated Oligo (SEQ ID NO: 9)), 11 and 13 were used. For the preparation of Y_{R}, SEQ ID NO: 2, 3 (and their respective modified versions; same as for the case of Y_{L}) and 14 were used.

The sequences of the linear nucleic acids used in the example of the present disclosure are as shown in the following Table 1, and parts of the short sequences which may bind with ribonucleic acids in a hairpin loop structure were shown in italic font.

TABLE 1

| | SEQ | |
|---|---|---|
| | ID | |
| Sequence (5′-3′) | NO | Note |
| AGGCTGATTCGGTTCATGCGGATCCA | 1 | — |
| TGGATCCGCATGACATTCGCCGTAAG | 2 | — |
| CTTACGGCGAATGACCGAATCAGCCT | 3 | — |
| /Biotin/AGGCTGATTCGGTTCATGCGGATCCA | 4 | SEQ ID NO: 1 |
| | | modification |
| /6-FAM/TGGATCCGCATGACATTCGCCGTAAG | 5 | SEQ ID NO: 2 |
| | | modification |
| /6-FAM/CTTACGGCGAATGACCGAATCAGCCT | 6 | SEQ ID NO: 3 |
| | | modification |
| /Cy5/TGGATCCGCATGACATTCGCCGTAAG | 7 | SEQ ID NO: 2 |
| | | modification |
| /Cy5/CTTACGGCGAATGACCGAATCAGCCT | 8 | SEQ ID NO: 3 |
| | | modification |
| /Biotin/TGGATCCGCATGACATTCGCCGTAAG | 9 | SEQ ID NO: 2 |
| | | modification |
| /AlexaFluor488/CTTACGGCGAATGACCGAATCA | 10 | SEQ ID NO: 3 |
| GCCT | | modification |
| /Fluorophoreylation/GACTCTTACGGCGAATGACC | 11 | SEQ ID NO: 3 |
| GAATCAGCCT | | modification |
| /Fluorophoreylation/AGTCAGGCTGATTCGGTTCA | 12 | SEQ ID NO: 1 |
| TGCGGATCCA | | modification |
| /Fluorophoreylation/GCATAGGCTGATTCGGTTCA | 13 | SEQ ID NO: 1 |
| TGCGGATCCA | | modification |
| /Fluorophoreylation/ATGCAGGCTGATTCGGTTCA | 14 | SEQ ID NO: 1 |
| TGCGGATCCA | | modification |
| /TEX615/TGGATCCGCATGACATTCGCCGTAAG | 15 | SEQ ID NO: 2 |
| | | modification |
| /TEX615/CTTACGGCGAATGACCGAATCAGCCT | 16 | SEQ ID NO: 3 |
| | | modification |
| /5IAbRQ/GCGAGGCCAGACTGGGAAGAAATCTGC | 17 | SEQ ID NO: 1 |
| TCGC/iCy5/AGGCTGATTCGGTTCATGCGGATCC | | modification |
| A | | |
| /5IAbRQ/GCGAGTCAACATCAGTCTGATAAGCTAC | 18 | SEQ ID NO: 1 |
| TCGC/iCy3/AGGCTGATTCGGTTCATGCGGATCC | | midification |
| A | | |

* i = internalized

A Y-shaped fluorescence labeled nucleic acid sphere in which code signals are concentrated on was prepared by coating a streptavidin-coated silica or polystyrene bead (manufactured by Bangs Laboratories, Inc.) with the fluorescence labeled nucleic acid structure prepared as described above through the streptavidin-biotin binding.

When fluorescence was observed using a confocal microscope, as shown in FIG. 3, a total of two fluorophores were determined to be capable of binding to the Y-shaped fluorescence labeled nucleic acid sphere, and a total of four fluorophores were determined to be capable of binding to branch-shaped fluorescence labeled nucleic acid sphere. From the result, when fluorophores having a blue color, a green color, and a red color were assigned respectively in consideration to the wavelength range of the emission spectrum of the fluorophore, and these three fluorophores are used, it was determined that total of 15 codes may be embodied (six codes corresponding to the two-fluorophore sphere, and nine codes corresponding to the four-fluorophore sphere).

Further, the present inventors personally designed an MATLAB computer language program as shown in the following Table 2 such that color codes may be predicted.

TABLE 2
% filename = ‘RB.png’; nx = 30; ny = 15;
cd(‘KSP_DNA nanobarcode’)
%%% B-3 G-2 R-1
Ms = meshgrid(1:100,1:100);
prompt = ‘Red - 1; Green - 2; Blue - 3; Please input in this format e.g.
[1 2 3] with spaces’;
result = input(prompt);
prompt2 = ‘Intensity e.g. 0.5 (should be between 0 and 1)’;
result2 = input(prompt2);
if ~isempty(result)
nR = length(find(result == 1));
nG = length(find(result == 2));
nB = length(find(result == 3));
nrgb = [nR nG nB];
nrgb = nrgb./max(nrgb)*result2;
nrgb = round(nrgb*2\hat{ }8);
Ms(:,:,1) = nrgb(1);
Ms(:,:,2) = nrgb(2);
Ms(:,:,3) = nrgb(3);
X = uint8(Ms);
figure(5); clf; imshow(X);
prompt = ‘Would you like to save this image? [Yes (9) No (0)]’;
ynres = input(prompt);
if ynres == 9
imwrite(X, [‘Im’ int2str(result) ‘.bmp’])
end
end
return;

Further, the present inventors personally designed a C++ computer language program as shown in the following Table 3 such that the number of signal codes may be predicted.

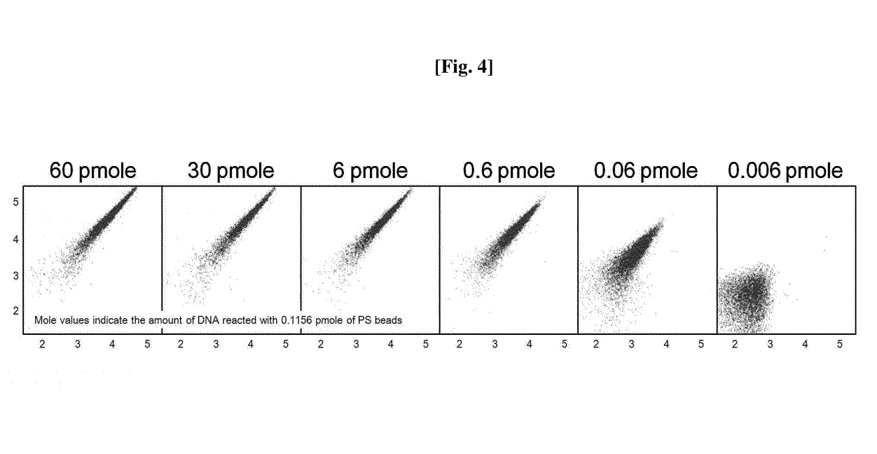

Further, an amount of fluorescence labeled nucleic acid structures which are bound to the silica or polystyrene bead was adjusted, and signal intensity was measured using a flow cytometer. Consequently, as shown in FIG. 4, it was determined that signal intensity sensed according to an amount of the fluorescence labeled nucleic acid structures may be adjusted.

### Example 2

### Preparation of Fusion Nano Liposome-Fluorescence Labeled Nucleic Acid

The present inventors adjusted a surface charge of the fusion nano liposome-fluorescence labeled nucleic acid by changing the lipid composition of the liposome to adjust interaction with cells according to types of the applied experiments. That is, when the fusion nano liposome-fluorescence labeled nucleic acid is prepared with a liposome formed with a lipid having a positive charge, the surface charge of the fusion nano liposome-fluorescence labeled nucleic acid becomes positive. Thus when a cell is treated with the fusion nano liposome-fluorescence labeled nucleic acid, the fusion nano liposome-fluorescence labeled nucleic acid is fused into a cell membrane non-specifically to cells, and the nucleic acid nanostructure according to an embodiment of the present disclosure flows into a cytoplasm. When a nucleic acid of the surface of the sphere flowing into a cytoplasm is a hairpin loop structured nucleic acid structure, the fusion nano liposome-fluorescence labeled nucleic acid may interact with a messenger ribonucleic acid (mRNA) or a micro ribonucleic acid (miRNA) in cells, and when a nucleic acid structure is a Y-shaped or branch-shaped nucleic acid structure, codes of various colors may be embodied in cells. Further, when the fusion nano liposome-fluorescence labeled nucleic acid is prepared with a liposome formed with a lipid having a neutral charge, non-specific binding of the fusion nano liposome-fluorescence labeled nucleic acid to types of cells may be reduced. To maximize the above-described effects, the lipid such as 18:1 PEG2000 PE was mixed to add a function of polyethylene glycol. Here, a cell luteal hormone releasing hormone-targeted protein was further bound to the surface of the liposome to specifically interact with a specific receptor (luteal hormone releasing hormone receptor) of the surface of the target cancer. Accordingly, the prepared fusion nano liposome-fluorescence labeled nucleic acid flows into cells through a luteal hormone releasing hormone receptor which is present at the surface of the cancer cell by receptor mediated endocytosis. A more specific method of producing the fusion nano liposome-fluorescence labeled nucleic acid is as follows.

2-1. Preparation of Liposome

To prepare the liposome having a positive charge, DOTAP (1,2-dioleoyl-3-trimethylammonium-propane) and cholesterol, which are cationic lipids having a positive charge and dissolved in chloroform, were mixed in the mass ratio of 7:3 to have a total mass of 2.5 mg, put in a glass bottle, and then dried in a vacuum drier for 16 hours to remove chloroform. Then, 1 ml of axenic distilled water was added to hydrate the dried lipids for 1 hour, at which time the glass bottle was shaken for 30 seconds one time every ten minutes such that a size of the liposome which is prepared through hydration was reduced to have a predetermined size or less. The prepared liposome passed through a porous polycarbonate filter having a pore size of 100 nm 20 times using an extruder such that the liposome was uniformized to have a size of 100 nm

Further, to prepare the liposome having a neutral charge, DOPC (1,2-dioleoyl-sn-glycero-3-phosphocholine) which is one of the neutral lipids, 18:1 PEG2000 PE (1,2-dioleoyl-sn-glycero-3-phosphoethanolamine-N-[methoxy(polyethylene glycol)-2000]) which is a lipid including polyethylene glycol, DOPE(1,2-dioleoyl-sn-glycero-3-phosphoethanolamine) which includes an amine functional group and may bind a targeted protein to the surface of the liposome later, and cholesterol were mixed in the mass ratio of 12:1:1:6 to have a total mass of 2.5 mg. The following production process was performed in a same manner as the liposome having a positive charge.

2-2. Preparation of Fusion Nano Liposome-Fluorescence Labeled Nucleic Acid

The liposome prepared in Example 2-1 and a nanosphere in which the fluorescence labeled nucleic acid structure prepared in Example 1 was bound to a surface, which were respectively included in solutions of axenic distilled water, were mixed in the volume ratio of 1:3. Here, solvents of the liposome and nanosphere should be the same such that a destruction of the liposome due to an osmotic pressure may be prevented. The mixed solution was reacted at room temperature for 1 hour, and mixed one time every 15 minutes using a pipet during a reaction (fusion reaction).

When the liposome having a positive charge was used for a reaction, the prepared fusion nano liposome-fluorescence labeled nucleic acid was sunken using a centrifuge after a fusion reaction, a supernatant was removed, the solution was cleaned twice using a 200 μl of a PBS solution, and then released to a final 100 μl of PBS solution.

When the liposome having a neutral charge was used for a reaction, the prepared fusion nano liposome-fluorescence labeled nucleic acid was sunken using a centrifuge after a fusion reaction, a supernatant was removed, 500 μl of SM(PEG)₂₄ (manufactured by Thermo Fisher Scientific Inc.) was added to the solution at a concentration of 3 mM as a linker for binding a luteal hormone releasing hormone (LHRH)-targeted protein, which is a targeted protein, to a surface of the liposome, and the solution was reacted at room temperature for 1 hour. After a reaction, the centrifuge was used again to sink the fusion nano liposome-fluorescence labeled nucleic acid, a supernatant was removed, 500 μl of LHRH-targeted protein, which is a targeted protein, was added to the solution at a concentration of 0.3 mM together with TCEP at a concentration of 5 mM, and then the solution was reacted at room temperature for 2 hours. After a reaction, the centrifuge was used again to sink the fusion nano liposome-fluorescence labeled nucleic acid, a supernatant was removed, the solution was cleaned twice using a 200 μl of a PBS solution, and then released to a final 100 μl of PBS solution.

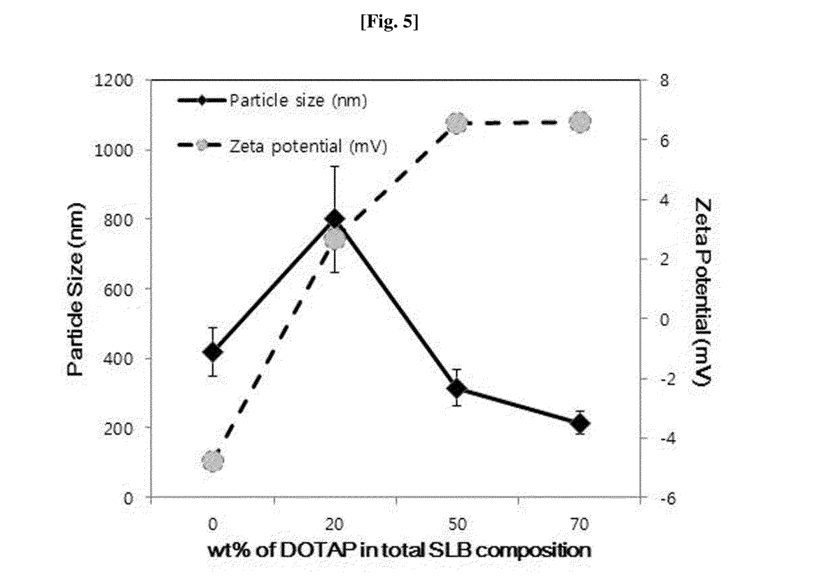

The fusion nanobarcode liposome-fluorescence labeled nucleic acid prepared as described above was measured using a transmission electron microscope (TEM) and dynamic light scattering (DLS) to analyze a particle size and surface charge properties. In the result of analysis using a DLS as shown in FIG. 5, it was determined that the surface charge of the fusion nano liposome-fluorescence labeled nucleic acid may be adjusted to a desired degree by adjusting the lipid composition of the liposome. In this regard, FIG. 5 illustrates a relationship of particle size (nm) and Zeta potential (mV) with respect to wt % of DOTAP in the total supported lipid bilayer (SLB) composition.

### Example 3

### Analysis of Fluorescence Nanobarcode Function Expression in Cells

To determine functions of the fusion nano liposome-fluorescence labeled nucleic acid prepared in Example 2 which is a fluorescence nanobarcode and the fusion nano liposome-fluorescence labeled hairpin loop structured nucleic acid, analysis was performed using a flow cytometer and a confocal microscope as follows.

3-1. Analysis of Fluorescence Nanobarcode Function Expression Using Flow Cytometer

After seeding breast cancer cells (MCF-7) on a 24 well plate, in consideration of a doubling time of cells, the breast cancer cells were cultured in an RPMI 1640 culture media including 10% of FBS such that confluency reached 90%. When the cells were cultured to have a target amount, the media was removed and cleaned using a PBS solution, 30 μl of a solution (solvent of axenic distilled water) including the fusion nano liposome-fluorescence labeled (hairpin loop structured) nucleic acid was mixed with the culture media such that the mixed solution had a volume of 1 ml. Then each well was treated with the mixed solution. Then, the cells are cultured at 37° C. for 15 minutes, 30 minutes, 1 hour, two hours, or four hours. The cells were collected using trypsin and moved to 1.5 ml-tubes for experiments when the culture was over, the solution was removed using a centrifuge, and the cells were cleaned three times using a PBS solution. Finally, the cells were treated with 100 μl of a 4% formaldehyde solution for 20 minutes and fixed, the fixed cells were maintained at 37° C. for five minutes, and then cooled to 4° C. using a thermocycler at a rate of −1° C./sec. Here, when a fluorescence labeled hairpin loop structured nucleic acid structure was not used, the use of the thermocycler was omitted.

The fixed cells which were obtained as described above were analyzed by measuring a fluorescence value using a flow cytometer. When a target receptor on a surface of the cell membrane was targeted, ovarian cancer cells (SK-OV-3) were used as a control group, when a messenger ribonucleic acid (mRNA) was detected, normal mammary cells (MCF-10A) were used, and when a micro ribonucleic acid (miRNA) was detected, a different type of breast cancer cells (SK-BR-3) was used as a control group.

As a result, as shown in FIG. 6, when an LHRH receptor was targeted, the fusion nano liposome-fluorescence labeled nucleic acid to which an LHRH targeted protein was additionally bound was determined to be successfully introduced into cells.

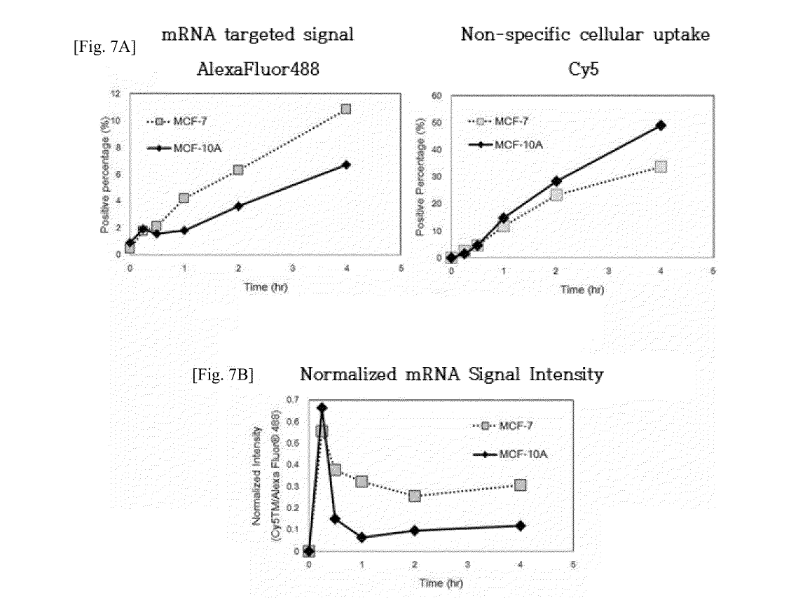

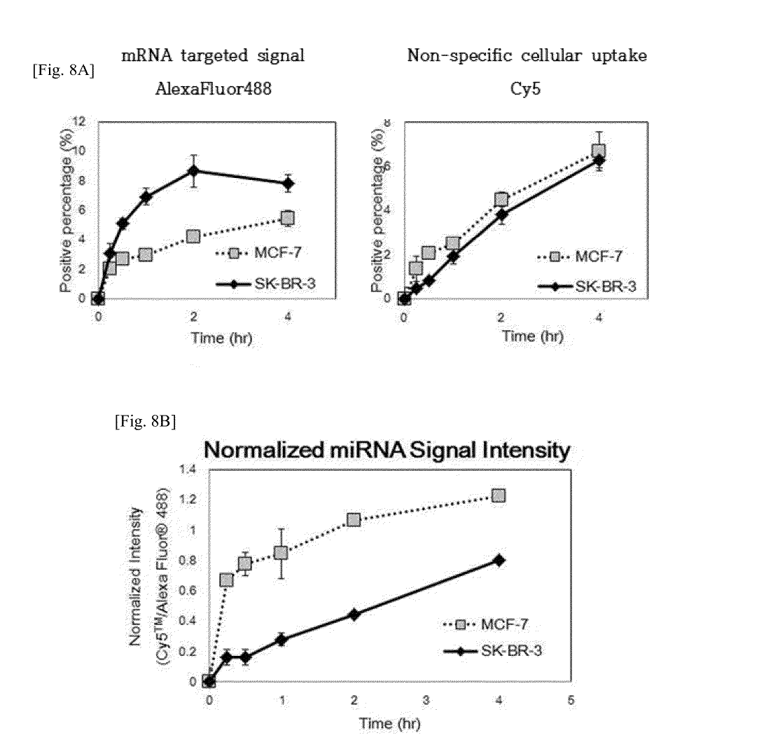

Further, when the internal signal of cancer cells was detected using EZH2 messenger ribonucleic acids, as shown in FIGS. 7A and 7B, a signal intensity of the fusion nano liposome-fluorescence labeled hairpin loop structured nucleic acid was determined to be excellent, and when the internal signal of cancer cells was detected using miR21 micro ribonucleic acids, as shown in FIGS. 8A and 8B, a signal intensity of the fusion nano liposome-fluorescence labeled hairpin loop structured nucleic acid was also determined to be excellent. Here, by comparing a ratio of the signal (e.g., Alexa (Fluor) 488) from fluorophores bound to another end of the Y-shaped nucleic acid structure to which the hairpin loop structure is connected in comparison with a fluorescence signal (e.g., Cy5) increasing according to a change to a linearized shape of the hairpin loop structured nucleic acid structure, diagnosis which is simple, rapid, and has an excellent selectivity as compared to conventional techniques is possible.

That is, since the degree of introduction of materials is different according to types of cells, when a signal generated from the hairpin loop structure is merely measured without determining an absolute quantity of materials which are introduced into cells, there is a problem in that distinction of cells is difficult. For example, when comparing the case in which 10 diagnostic substances are introduced into an arbitrary cell and 5 diagnostic substances among them go through a change to a linearized shape and the case in which 1,000 diagnostic substances are introduced into an arbitrary cell and 5 diagnostic substances among them go through a change to a linearized shape, the signal is only generated from 5 diagnostic substances in both of the cases, and thus two cells may not be distinguished without determining an entire amount of the introduced diagnostic substances. However, when comparing the ratio of the linearized diagnostic substances with respect to the entire amount of the introduced diagnostic substances, it may be compared as to which cell group has a larger amount of the target nucleic acids. Thus, according to an embodiment of the present disclosure, it is unnecessary to perform an optimization process based on types of cells which is required in a conventional system in which an absolute quantity of fluorescence generated from the diagnostic substances are merely measured. Further, a system is achieved in which high specificity may be embodied by using nucleic acids, and at the same time, target nucleic acids may be diagnosed in a short time after the diagnostic substance are introduced into cells (within about 30 minutes) such that nucleic acids may work in the cytoplasm.

3-2. Analysis of Expression of Fluorescence Nanobarcode Function Using Confocal Microscope

After seeding breast cancer cells (MCF-7) on a plate for cell culture, in consideration of a doubling time of cells, the breast cancer cells were cultured in an RPMI 1640 culture media including 10% of FBS such that confluency reached 90%. When the cells were cultured to have a target amount, the media was removed and cleaned using a PBS solution. Thereafter, 30 μl of a solution containing the fusion nano liposome-fluorescence labeled nucleic acids was mixed with the medium such that the mixed solution had a volume of 1 ml, each well was treated with the mixed solution, and then the cells are cultured at 37° C. for 15 minutes, 30 minutes, 1 hour, two hours, or four hours. After the culture, the cells were cleaned three times using a PBS solution and determined using a confocal microscope. Here, when a micro ribonucleic acid (miRNA) was detected, a different type of breast cancer cells (SK-BR-3) was used as a control group, when a messenger ribonucleic acid (mRNA) was detected, normal mammary cells (HMEC) were used, and when a target receptor on a surface of the cell membrane was targeted, ovarian cancer cells (SK-OV-3) were used as a control group. As a result, like the results of Example 3-1, a signal intensity of the fusion nano liposome-fluorescence labeled nucleic acid was determined to be successfully introduced into cells and thereby fluorescence was observed with an excellent brightness in cancer cells.

The above description of the present disclosure was merely for an example, and it will be apparent to those skilled in the art that a modification to another particular form can be easily made without departing from the spirit and essential feature of the present disclosure. Accordingly, the examples described above should be understood to be exemplary in all aspects, and not restrictive.

The fusion nano liposome-fluorescence labeled nucleic acid according to an embodiment of the present disclosure may sense an external or internal signal according to the use of a targeted protein or hairpin loop structured nucleic acid structure, and thus disease-specific diagnosis is possible. Further, highly-sensitive diagnosis is possible even when a messenger ribonucleic acid (RNA) and micro RNA which are present at an absolutely low concentration in cells being targeted. Thus initial cancer of which targeted materials are less expressed may be diagnosed, various target materials expressed inside and outside of the cell membrane may be targeted, and thus even a type of cancer which is hard to diagnose such as triple negative breast cancer or the like may also be flexibly diagnosed. Further, a fusion nano fluorescence labeled nucleic acid is allowed to correspond to a type of diseases by using various fluorophores, and thus it is possible to achieve multiplexed detection in which multiple diseases may be diagnosed at the same time. Further, a liposome bilayer on a surface of the fusion nano liposome-fluorescence labeled nucleic acid decreases a decomposition rate of the diagnostic substance in cells, and thus the in vivo application is facilitated. In addition, the nucleic acid is used to diagnose a targeted material, and thus selectivity is high. Nucleic acid sequences are changed according to a target mRNA and miRNA, and thus a wide range of the flexible diagnosis of disease is possible.

It will be apparent to those skilled in the art that various modifications can be made to the above-described exemplary embodiments of the present disclosure without departing from the spirit or scope of the present disclosure. Thus, it is intended that the present disclosure covers all such modifications provided they come within the scope of the appended claims and their equivalents.

What is claimed is:

1. A fusion nano liposome-fluorescence labeled nucleic acid for diagnosis, in which a polystyrene or silica bead having a surface binding with a branch-shaped nucleic acid structure labeled with a fluorophore is included in an inside of a liposome.

2. The fusion nano liposome-fluorescence labeled nucleic acid of claim 1, wherein the nucleic acid structure has a shape in which linear nucleic acids selected from the group consisting of a base sequence of SEQ ID NO: 1 to 3 are bound to be in a Y-branched shape.

3. The fusion nano liposome-fluorescence labeled nucleic acid of claim 2, wherein the linear nucleic acid further comprises a fluorophore at a 5′ end.

4. The fusion nano liposome-fluorescence labeled nucleic acid of claim 3, wherein the fluorophore is selected from the group consisting of fluorescein, Texas Red, rhodamine, alexa, cyanine, BODIPY, and coumarin.

5. A fusion nano liposome-fluorescence labeled hairpin loop structured nucleic acid for diagnosis, in which a polystyrene or silica bead having a surface binding with a branch-shaped nucleic acid structure having a hairpin loop end is included in an inside of a liposome.

6. The fusion nano liposome-fluorescence labeled hairpin loop structured nucleic acid of claim 5, wherein the hairpin loop end comprises a base sequence having a complementary sequence with a target ribonucleic acid (RNA).

7. The fusion nano liposome-fluorescence labeled hairpin loop structured nucleic acid of claim 5, wherein the nucleic acid structure is further labeled with a fluorophore and a quencher.

8. The fusion nano liposome-fluorescence labeled hairpin loop structured nucleic acid of claim 7, wherein the fluorophore is selected from the group consisting of fluorescein, Texas Red, rhodamine, alexa, cyanine, BODIPY, and coumarin.

9. The fusion nano liposome-fluorescence labeled hairpin loop structured nucleic acid of claim 7, wherein the quencher is selected from the group consisting of TAMRA, BHQ, Iowa Black RQ, and a molecular grove binding non-fluorescence quencher (MGBNFQ).

10. The fusion nano liposome-fluorescence labeled hairpin loop structured nucleic acid of claim 5, wherein the branch-shaped nucleic acid structure having a hairpin loop end has a shape in which linear nucleic acids are bound to be in a Y-branched shape, and one or more of the linear nucleic acids form a hairpin loop end.

11. The fusion nano liposome-fluorescence labeled nucleic acid of claim 1, wherein the liposome is formed of a cationic lipid including DOTAP (1,2-dioleoyl-3-trimethylammonium-propane) and cholesterol as constituents.

12. The fusion nano liposome-fluorescence labeled nucleic acid of claim 1, wherein the liposome is formed of a neutral lipid including DOPC (1,2-dioleoyl-sn-glycero-3-phosphocholine), 18:1 PEG2000 PE (1,2-dioleoyl-sn-glycero-3-phosphoethanolamine-N-[methoxy(polyethylene glycol)-2000]), DOPE (1,2-dioleoyl-sn-glycero-3-phosphoethanolamine), and cholesterol as constituents.

13. The fusion nano liposome-fluorescence labeled hairpin loop structured nucleic acid of claim 5, wherein the liposome is formed of a cationic lipid including DOTAP (1,2-dioleoyl-3-trimethylammonium-propane) and cholesterol as constituents.

14. The fusion nano liposome-fluorescence labeled hairpin loop structured nucleic acid of claim 5, wherein the liposome is formed of a neutral lipid including DOPC (1,2-dioleoyl-sn-glycero-3-phosphocholine), 18:1 PEG2000 PE (1,2-dioleoyl-sn-glycero-3-phosphoethanolamine-N-[methoxy(polyethylene glycol)-2000]), DOPE (1,2-dioleoyl-sn-glycero-3-phosphoethanolamine), and cholesterol as constituents.

15. A bio-diagnostic imaging system comprising the fusion nano liposome-fluorescence labeled nucleic acid of claim 1.

16. The bio-diagnostic imaging system of claim 15, wherein the system measures fluorescence in cells.

17. A bio-diagnostic imaging system comprising the fusion nano liposome-fluorescence labeled hairpin loop structured nucleic acid of claim 5.

18. The bio-diagnostic imaging system of claim 17, wherein the system measures fluorescence in cells.

19. A method of producing a fusion nano liposome-fluorescence labeled nucleic acid for diagnosis, comprising the following steps:
a) preparing a Y-branch-shaped nucleic acid structure with linear nucleic acids which respectively have a fluorophore, biotin, or cohesive end at a 5′ end using an annealing method;
b) binding the nucleic acid structure to a streptavidin-coated surface of a polystyrene or silica bead to prepare a fluorescence-labeled nucleic acid nanosphere; and
c) mixing a solution containing the fluorescence-labeled nucleic acid nanosphere, and a solution containing a liposome formed of a cationic lipid or neutral lipid.

20. The method of claim 19, wherein the linear nucleic acid is selected from the group consisting of a base sequence of SEQ ID NO: 1 to 16.

21. The method of claim 19, wherein the fluorophore is selected from the group consisting of fluorescein, Texas Red, rhodamine, alexa, cyanine, BODIPY, and coumarin.

22. The method of claim 19, wherein the liposome formed of a cationic lipid is prepared by mixing DOTAP (1,2-dioleoyl-3-trimethylammonium-propane) and cholesterol in the mass ratio of 6:4 to 9:1.

23. The method of claim 19, wherein the liposome formed of a neutral lipid is prepared by mixing DOPC (1,2-dioleoyl-sn-glycero-3-phosphocholine), 18:1 PEG2000 PE (1,2-dioleoyl-sn-glycero-3-phosphoethanolamine-N-[methoxy(polyethylene glycol)-2000]), DOPE (1,2-dioleoyl-sn-glycero-3-phosphoethanolamine), and cholesterol in the mass ratio of 12:1:1:6 to 14:1:1:1.

24. The method of claim 19, wherein the method further comprises preparing a branch-shaped nucleic acid structure by connecting a cohesive end of a Y-branch-shaped nucleic acid structure to another Y-branch-shaped nucleic acid structure using a T4 ligase after step a).

25. The method of claim 19, wherein a solution containing the sphere and a solution containing the liposome are mixed in the volume ratio of 1:1 to 1:4 in step c).

26. A method of producing a fusion nano liposome-fluorescence labeled hairpin loop structured nucleic acid for diagnosis, comprising the following steps:
a) preparing a Y-branch-shaped nucleic acid structure with linear nucleic acids which respectively have a fluorophore and quencher, biotin, or cohesive end at a 5′ end using an annealing method, wherein one or more of the linear nucleic acids include a base sequence having a complementary sequence with a target RNA, and a base sequence forming a hairpin loop end at the 5′ end;
b) binding the hairpin loop structured nucleic acid structure to a streptavidin-coated surface of a polystyrene or silica bead to prepare a fluorescence labeled hairpin loop structured nucleic acid nanosphere; and
c) mixing a solution containing the fluorescence labeled hairpin loop structured nucleic acid nanosphere, and a solution containing a liposome formed of a cationic lipid or neutral lipid.

27. The method of claim 26, wherein the linear nucleic acid is selected from the group consisting of a base sequence of SEQ ID NO: 17 or 18.

28. The method of claim 26, wherein the quencher is selected from the group consisting of TAMRA, BHQ, Iowa Black RQ, and a molecular grove binding non-fluorescence quencher (MGBNFQ).

29. The method of claim 26, wherein the fluorophore is selected from the group consisting of fluorescein, Texas Red, rhodamine, alexa, cyanine, BODIPY, and coumarin.

30. The method of claim 26, wherein the liposome formed of a cationic lipid is prepared by mixing DOTAP (1,2-dioleoyl-3-trimethylammonium-propane) and cholesterol in the mass ratio of 6:4 to 9:1.

31. The method of claim 26, wherein the liposome formed of a neutral lipid is prepared by mixing DOPC (1,2-dioleoyl-sn-glycero-3-phosphocholine), 18:1 PEG2000 PE (1,2-dioleoyl-sn-glycero-3-phosphoethanolamine-N-[methoxy(polyethylene glycol)-2000]), DOPE (1,2-dioleoyl-sn-glycero-3-phosphoethanolamine), and cholesterol in the mass ratio of 12:1:1:6 to 14:1:1:1.

32. The method of claim 26, wherein the method further comprises preparing a branch-shaped nucleic acid structure by connecting a cohesive end of a Y-branch-shaped nucleic acid structure to another Y-branch-shaped nucleic acid structure using a T4 ligase after step a).

33. The method of claim 26, wherein a solution containing the sphere and a solution containing the liposome are mixed in the volume ratio of 1:1 to 1:4 in step c).

34. A method of diagnosing disease, comprising
a step of injecting the fusion nano liposome-fluorescence labeled nucleic acid of claim 1 to a subject requiring diagnosis of disease, and measuring fluorescence.

35. The method of claim 34, wherein the disease is cancer.

36. A method of diagnosing disease, comprising
a step of injecting the fusion nano liposome-fluorescence labeled hairpin loop structured nucleic acid of claim 5 to a subject requiring diagnosis of disease, and measuring fluorescence.

37. The method of claim 36, wherein the disease is cancer.
